(19) United States
(12) Patent Application Publication
(10) Pub. No.: US 2023/0131843 A1
(43) Pub. Date: Apr. 27, 2023

(54) UNIVERSAL AND LIQUID EXPANSION TYPE CLAMPING DEVICE FOR PELVIC FRACTURE REDUCTION ROBOT

(71) Applicant: Shanghai University, Shanghai (CN)

(72) Inventors: Jingtao Lei, Shanghai (CN); Xinyi Chen, Shanghai (CN); Shenyang Cai, Shanghai (CN)

(21) Appl. No.: 17/957,730

(22) Filed: Sep. 30, 2022

(30) Foreign Application Priority Data
Oct. 22, 2021 (CN) 202111229813.4

(51) Int. Cl. A61B 17/64

(52) U.S. Cl. CPC A61B 17/6433; A61B 17/6408

(57) ABSTRACT

The present disclosure relates to a universal and liquid expansion type clamping device for a pelvic fracture reduction robot. The clamping device is composed of an affected side spherical hinge support module, a pin rod clamping part, a rod-rod clamping part, a butt joint module, and a healthy side fixing module. The affected side spherical hinge support module is a main module for the pelvis clamping device, and by adopting a spherical hinge structure, the affected side spherical hinge support module is connected to the pin rod clamping part and the butt joint module respectively. The rod-rod clamping part is fixedly installed on one side of an operation table by the healthy side fixing module.

### CROSS REFERENCE TO RELATED APPLICATION

This patent application claims the benefit and priority of Chinese Patent Application No. 202111229813.4, filed on Oct. 22, 2021, the disclosure of which is incorporated by reference herein in its entirety as part of the present application.

### TECHNICAL FIELD

The present disclosure relates to the technical field of medical robots, and in particular to a universal and liquid expansion type clamping device for a pelvic fracture reduction robot.

### BACKGROUND ART

Pelvic fracture is one of the most challenging fractures in traumatic orthopedic clinics due to its high mortality rate and high incidence of concurrent injuries. Due to the complexity of pelvic anatomy, pelvic fractures are divided into three types, namely, A, B, and C, according to Tile classification, where Tile B and C are classified as unstable pelvic fractures. Secondary injuries and surgical complications are easy to occur in the reduction operation of unstable pelvic fractures, resulting in the death and disability rate of unstable pelvic fractures as high as 10% to 50%.

With the development of robotics, robot surgery is expected to replace traditional surgery, which has the advantages of minimally invasive, high surgical accuracy, quick recovery of patients after surgery, light operation intensity of doctors, less radiation exposure to both doctors and patients, etc.

Robot pelvic fracture reduction surgery is applicable to B-type or C-type unstable fractures with displacement and rotation deviation. Preoperative stabilization of the injured pelvis with a clamping device at the tail end of the robot is required. Firstly, as the space between the robot and the injured pelvic tissue is very small, the clamping device needs to meet the requirement of compact structure. Secondly, as the reduction force for pelvic fracture reduction is as high as 500 N, the clamping device not only needs to meet the requirements of rigidity and strength, but also should meet the requirement of clamping stability of the pelvis. As the anatomical parameters of the pelvis are different in patients of different genders and ages, the clamping device needs to adapt to personalized pelvis size and pelvic fracture type of different patients. At present, there is a lack of systematic and in-depth research on the clamping device for robot pelvic fracture reduction.

To solve the technical problems above, the present disclosure relates to a universal and liquid expansion type clamping device for a pelvic fracture reduction robot, which can adapt to pins at different spatial positions and postures according to the personalized pelvis and injury types of the patients so as to achieve stable clamping of the pelvis.

### SUMMARY

To solve the defects in the prior art, the present disclosure provides a universal and liquid expansion type clamping device for a pelvic fracture reduction robot. The clamping device can adapt to pins at different spatial positions according to the personalized pelvis and injury types, thereby achieving stable clamping of the pelvis.

To achieve the objective above, the present disclosure adopts the following technical solutions:

A universal and liquid expansion type clamping device for a pelvic fracture reduction robot includes an affected side spherical hinge support module, a pin rod clamping part, a rod-rod clamping part, a butt joint module, and a healthy side fixing module. The affected side spherical hinge support module is a main module for the pelvic clamping device, and by adopting a spherical hinge structure, the affected side spherical hinge support module is connected to the pin rod clamping part and the butt joint module respectively. The rod-rod clamping part is fixedly installed on the side of an operation table by the healthy side fixing module. The working principle is described as follows: the adjustment flexibility of the clamping device is improved by adopting the spherical hinge structure in the affected side spherical hinge support module, a liquid expansion structure in the pin rod clamping part and multi-directional adjustment structures of the pin rod clamping part and rod-rod clamping part. The clamping device can adapt to pins at different spatial positions and postures according to the personalized pelvis and pelvic injury types of different patients, the adjustment is convenient, and stable clamping can be achieved. The clamping device has the advantages of compact structure, flexible adjustment, high rigidity, firm fixation, easy assembly and disassembly and the like.

The affected side spherical hinge support module includes a spherical hinge base, a spherical hinge stop pin, a spherical hinge stopper, a spherical hinge cover plate and a spherical hinge cross rod. After the spherical hinge stop pin is placed in the spherical hinge base, the spherical hinge stopper can be adjusted by rotating the spherical hinge stop pin. The spherical hinge stopper, after being adjusted by the spherical hinge stop pin, can limit the spherical hinge cross rod with the spherical hinge cover plate. The spherical hinge cover plate is fixedly connected to the spherical hinge base. The adjustment flexibility of the clamping device is improved by adjusting an angle between the spherical hinge cross rod and the spherical hinge base by the affected side spherical hinge support module.

The pin rod clamping part includes a pin, a bolt, a pin clamping assembly, a spring, a first cross rod clamping assembly, two compression bolts, and a nut. The pin clamping assembly is coaxially connected to the first cross rod clamping assembly by the bolt. Hydraulic oil is packaged in the pin clamping assembly, and the inner wall of the clamping part is expanded by screwing the compression bolts to achieve the effect of clamping the pin. The first cross rod clamping assembly has basically the same structure as the bone needle clamping assembly and is used for clamping the cross bar, and the pin and the cross rod are fixed by screwing the bolt and the nut. The spring is arranged in a counter bore of the contact surface of the first cross bar clamping assembly and the pin clamping assembly. During clamping, the clamping stability is able to be guaranteed under the combined action of a meshing contact of tooth grooves of the contact surface and the elastic potential energy of the spring.

The rod-rod clamping part includes a bolt, a second cross rod clamping assembly, a spring, and a third cross rod clamping assembly. The second cross rod clamping assembly is coaxially connected to the third cross rod clamping assembly by a bolt, wherein the second cross rod clamping assembly is provided with textures at the joint with the cross rod to increase the friction during clamping. The third cross rod clamping assembly has the basically same structure as the second cross rod clamping assembly. During operation, the medical staff can fix the cross rod by screwing the third cross rod clamping assembly and the bolt. The spring is arranged in a counterbore of the contact surface of the second cross rod clamping assembly and the third cross rod clamping assembly. During clamping, the clamping stability is able to be guaranteed under the combined action of the meshing contact of the tooth grooves of the contact surface and the elastic potential energy of the spring.

The butt joint module includes a male connector, a female connector and two angular aluminum parts. The male connector is fixed to the tail end of the fracture reduction robot by means of a threaded connection. The female connector is connected to the male connector in order to apply hydraulic pressure to the male connector for fixation. The angular aluminum parts are fixed to the female connector and the affected side spherical hinge support module by means of a threaded connection. The butt joint module is used for rapidly connecting the tail end of the fracture reduction robot to the affected side support to form a pelvic fracture reduction system.

The healthy side fixing module includes two healthy side cross rods, a healthy side adjustable support, a healthy side fixing frame base, two healthy side fixing frame gaskets, two healthy side fixed frame rotary knobs, pin rod clamping parts and rod-rod clamping parts. The healthy side cross rods are fixed to the healthy side adjustable support by the rod-rod clamping parts. The pins placed in the anterior superior iliac spine and anterior inferior iliac spine on the healthy side of the pelvis are fixed by the pin rod clamping parts. The healthy side adjustable support is provided with equally spaced through holes and can be lifted and adjusted with the healthy side fixing frame base. The two healthy side fixing frame gaskets are pressed by the healthy side fixing frame base by rotating the healthy side fixing frame rotary knobs, such that the healthy side fixing frame base is fixed to the side of the operation table. By adjusting the height of the healthy side adjustable support and an angle of the healthy side cross rod, the healthy side fixing module is able to adapt to pins at different spatial positions and postures on the healthy side of the pelvis.

Compared with the prior art, the present disclosure has the following apparent outstanding substantive features and significant advantages:

The clamping device is used to fix the affected part of the pelvis, and the spherical hinge structure is used to fix the healthy part of the pelvis. By adopting the lifting structure, the clamping device can be conveniently adjusted, and it is convenient to adapt to the pins at different spatial positions and postures according to the personalized pelvic physiological structure and pelvic fracture type of the patients, thus achieving the stable clamping of the pelvis. By adopting the liquid expansion principle, the inner wall of the pin rod clamping part uniformly expands in an axial direction to guarantee the firm clamping of the pin and the uniform force on the pin and the cross rod. The butt joint module facilitates the rapid connection of the clamping device and the reduction robot. The clamping device has a number of obvious advantages including that of having compact structure, flexible adjustment, large rigidity, firm fixation and easy assembly and disassembly and the like.

### BRIEF DESCRIPTION OF THE DRAWINGS

FIG. 1 is a schematic diagram of an overall structure of a universal and liquid expansion type clamping device for a pelvic fracture reduction robot in accordance with the present disclosure.

FIG. 2 is a structure diagram of an affected side spherical hinge support module in accordance with the present disclosure.

FIG. 3 is a structure diagram of a pin rod clamping part in accordance with the present disclosure.

FIG. 4 is a structure diagram of a rod-rod clamping part in accordance with the present disclosure.

FIG. 5 is a structure diagram of a butt joint module in accordance with the present disclosure.

FIG. 6 is a structure diagram of a healthy side fixing module in accordance with the present disclosure.

### DETAILED DESCRIPTION OF THE EMBODIMENTS

The present disclosure will be further explained with reference to the following drawings and preferred embodiments. It should be understood that the following specific embodiments are only used to illustrate the present invention, but are not intended to limit the scope of the present disclosure.

### Embodiment I

Refer to FIG. 1, a universal and liquid expansion type clamping device for a pelvic fracture reduction robot includes an affected side spherical hinge support module 1, a pin rod clamping part 2, a rod-rod clamping part 3, a butt joint module 4, and a healthy side fixing module 5. The affected side spherical hinge support module 1 is a main module for the pelvic clamping device, and by adopting a spherical hinge structure, the affected side spherical hinge support module 1 is connected to the pin rod clamping part 2 and the butt joint module 4 respectively. The rod-rod clamping part 3 is fixedly installed on the side of an operation table by the healthy side fixing module 5.

In accordance with the universal and liquid expansion type clamping device for the pelvic fracture reduction robot, the adjustment flexibility of the clamping device is improved by adopting the spherical hinge structure in the affected side spherical hinge support module 1, a liquid expansion structure in the pin rod clamping part 2 and multi-directional adjustment structures of the pin rod clamping part 2 and rod-rod clamping part 3. The clamping device is able to adapt to pins at different spatial positions and postures according to the personalized pelvis and pelvic injury types of different patients, the adjustment is convenient, and the stable clamping is achieved. The clamping device has the advantages of compact structure, flexible adjustment, high rigidity, firm fixation, easy assembly and disassembly and the like.

### Embodiment II

As shown in FIG. 1, a universal and liquid expansion type clamping device for a pelvic fracture reduction robot includes an affected side spherical hinge support module 1, pin rod clamping parts 2, rod-rod clamping parts 3, a butt joint module 4, and a healthy side fixing module 5. The affected side spherical hinge support module 1 is a main module for the pelvic clamping device, and by adopting a spherical hinge structure, the affected side spherical hinge support module 1 is connected to the pin rod clamping part 2 and the butt joint module 4, respectively. The pin rod clamping part 2, after firmly clamping pins at the anterior superior iliac spine and anterior inferior iliac spine on the affected side of the pelvis, is fixedly connected to cross rods 11 and 15 of the affected side support. The pin rod clamping parts 2, after firmly clamping the anterior superior iliac spine and anterior inferior iliac spine on the healthy side of the pelvis, are also fixedly connected to the healthy side cross rods 51. The rod-rod clamping parts 3 are used to connect the healthy side cross rod 51 and the healthy side adjustable support 52. The butt joint module 4 is used to rapidly connect the affected side spherical hinge support module 1 to a surgical robot. The pelvis on the healthy side is fixed by the fixing module 5, and after the pins placed into the pelvis on the healthy side and the cross rods 51 are firmly clamped by the pin rod clamping parts 2, the firm fixation of the cross rods 51 and the healthy side adjustable support 52 is achieved using the rod-rod clamping parts 3. The adjustment flexibility of the clamping device is improved by adopting the spherical hinge structure in the affected side spherical hinge support module 1, a liquid expansion structure in the pin rod clamping part 2 and multi-directional adjustment structures of the pin rod clamping part 2 and rod-rod clamping part 3. The clamping device is able to adapt to pins at different spatial positions and postures according to the personalized pelvis and pelvic injury types of different patients, the adjustment is convenient, and stable clamping is achieved. The clamping device has obvious advantages of compact structure, flexible adjustment, high rigidity, firm fixation, easy assembly and disassembly and the like.

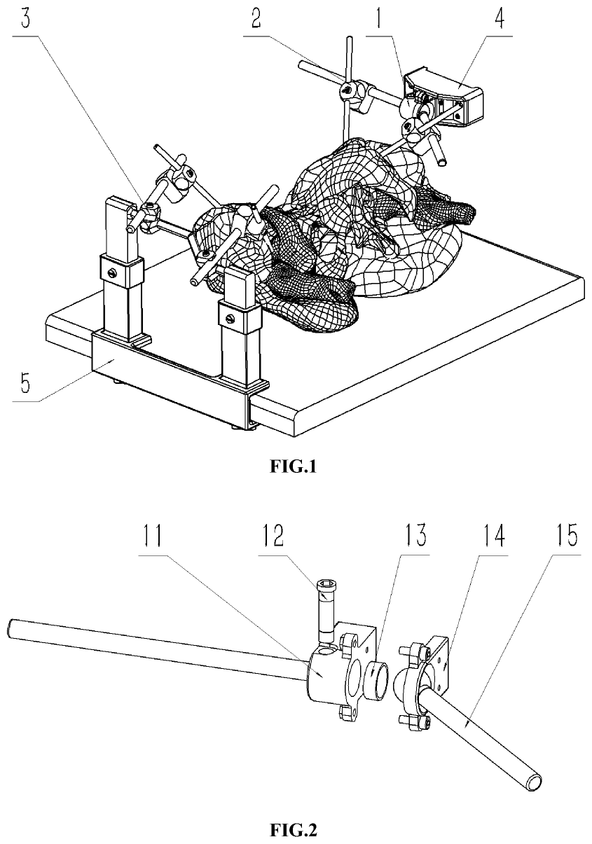

As shown in FIG. 2, the affected side spherical hinge support module 1 includes a spherical hinge base 11, a spherical hinge stop pin 12, a spherical hinge stopper 13, a spherical hinge cover plate 14 and a spherical hinge cross rod 15. After the spherical hinge stop pin 12 is placed in the spherical hinge base 11, the spherical hinge stopper 13 is able to be adjusted by rotating the spherical hinge stop pin 12. The spherical hinge stopper 13, after being adjusted by the spherical hinge stop pin 12, may limit the spherical hinge cross rod 15 with the spherical hinge cover plate 14. The spherical hinge cover plate 14 is fixedly connected to the spherical hinge base 11. The adjustment flexibility of the clamping device is improved by adjusting an angle between the spherical hinge cross rod 15 and the spherical hinge base 11 by the affected side spherical hinge support module 1.

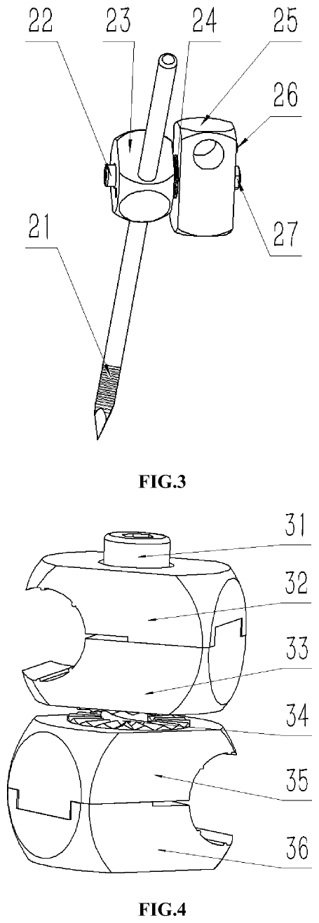

As shown in FIG. 3, the pin rod clamping part 2 includes a pin 21, a bolt 22, a pin clamping assembly 23, a spring 24, a first cross rod clamping assembly 25, two compression bolts 26, and a nut 27. The pin clamping assembly 23 is coaxially connected to the first cross rod clamping assembly 25 by the bolt 22. Hydraulic oil is packaged in the pin clamping assembly 23, and the inner wall of the clamping part is expanded by screwing the compression bolts 26 to achieve the effect of clamping the pin 21. The first cross rod clamping assembly 25 has basically the same structure as the bone needle clamping assembly 23 and is used for clamping, the cross bar, and the pin 21 and the cross rod are fixed by screwing the bolt 22 and the nut 27. The spring 24 is arranged in a counterbore of the contact surface of the first cross bar clamping assembly 25 and the pin clamping assembly 23. During clamping, the clamping stability is able to be guaranteed under the combined action of the meshing contact of the tooth grooves of the contact surface and the elastic potential energy of the spring 24.

As shown in FIG. 4, the rod-rod clamping part includes a bolt 31, a second cross rod clamping assembly 32, 33, a spring 34, and a third cross rod clamping assembly 35, 36. The second cross rod clamping assembly 32, 33 is coaxially connected to the third cross rod clamping assembly 35, 36 by a bolt 31. The second cross rod clamping assembly 32, 33 is provided with textures at the joint with the cross rod to increase the friction during clamping. The third cross rod clamping assembly 35, 36 has basically the same structure as the second cross rod clamping assembly 32, 33. During operation, the medical staff may fix the cross rod by screwing the third cross rod clamping assembly 35, 36 and the bolt 31. The spring 34 is arranged in a counterbore of the contact surface of the second cross rod clamping assembly 32, 33 and the third cross rod clamping assembly 35, 36. During clamping, clamping stability can be guaranteed under the combined action of the meshing contact of the tooth grooves of the contact surface and the elastic potential energy of the spring 34.

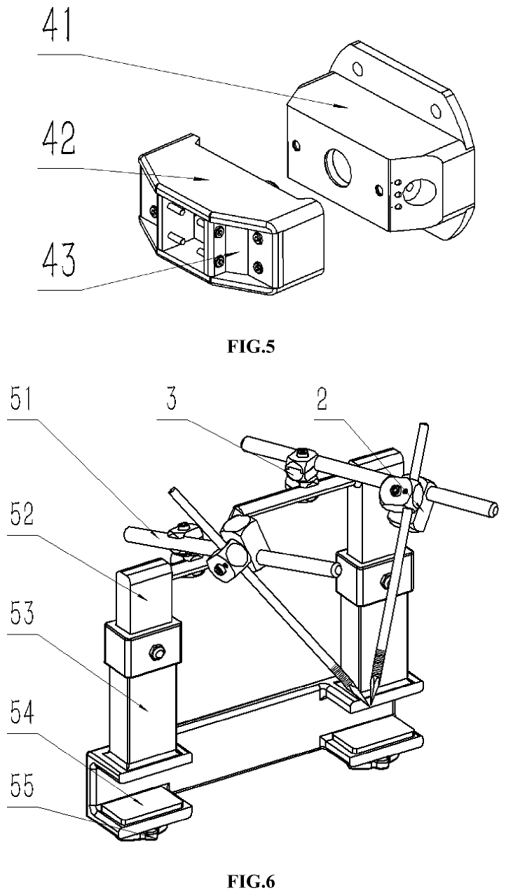

As shown in FIG. 5, the butt joint module 4 includes a male connector 41, a female connector 42 and two angular aluminum parts 43. The male connector 41 is fixed to the tail end of the fracture reduction robot by means of a threaded connection. The female connector 42 is connected to the male connector 41 to apply hydraulic pressure to the male connector for fixation. The angular aluminum parts 43 are fixed to the female connector 42 and the affected side spherical hinge support module 1 by means of a threaded connection. The butt joint module 4 is used for rapidly connecting the tail end of the fracture reduction robot to the affected side support 1 to form a pelvic fracture reduction system.

As shown in FIG. 6, the healthy side fixing module 5 includes two healthy side cross rods 51, a healthy side adjustable support 52, a healthy side fixing frame base 53, two healthy side fixing frame gaskets 54, two healthy side fixed frame rotary knobs 55, pin rod clamping parts 2 and rod-rod clamping parts 3. The healthy side cross rods 51 are fixed to the healthy side adjustable support 52 by the rod-rod clamping parts 3. The pins placed in the anterior superior iliac spine and anterior inferior iliac spine on the healthy side of the pelvis are fixed by the pin rod clamping parts 2. The healthy side adjustable support 52 is provided with equally spaced through holes and is able to be lifted and adjusted with the healthy side fixing frame base 53. The two healthy side fixing frame gaskets 54 are pressed by the healthy side fixing frame base 53 by rotating the healthy side fixing frame rotary knobs 55, such that the healthy side fixing frame base is fixed to the side of the operation table. By adjusting the height of the healthy side adjustable support 52 and an angle of the healthy side cross rod 5, the healthy side fixing module may adapt to pins at different spatial positions and postures on the healthy side of the pelvis.

The working principle of the present disclosure is described as follows:

(1) Respectively placing the pins 21 into the anterior superior iliac spine and the anterior inferior iliac spine of the affected side of the injured pelvis, and respectively placing the pins 21 into the anterior superior iliac spine and the anterior inferior iliac spine of the healthy side of the injured pelvis;

(2) fixing the healthy side fixing frame base 53 to one side of the operation table, adjusting the healthy side adjustable support 52 to an appropriate height, connecting, by the rod-rod clamping parts 3, the healthy side adjustable support 52 to the healthy side cross rods 51, adjusting the healthy side adjustable support 52 and the healthy side cross rods 51 to the appropriate positions, connecting, by the pin rod clamping parts 2, the pins 21 to the healthy side cross rods 51, and then screwing and checking the firmness of the rod-rod clamping parts 3 and the pin rod clamping parts 2 on the healthy side;

(3) adjusting an angle between the spherical hinge cross rod 15 and the spherical hinge base 11 in the affected side support 1, connecting, by the pin rod clamping part 2, the pins placed into the anterior superior iliac spine and anterior inferior iliac spine of the affected side of the pelvis to the affected side support 1, and screwing and checking the firmness of the pin rod clamping part 2 on the healthy side.

(4) After the pelvis is firmly fixed, connecting, by the male connector 41 connected to the tail end of the robot, the reduction robot to the female connector 42 in a butt-joint manner.

The present disclosure has the obvious advantages that the clamping device is used to fix the affected part of the pelvis, and the spherical hinge structure is used to fix the healthy part of the pelvis. By adopting a lifting structure, the clamping device can be conveniently adjusted, and it is convenient to adapt to the pins at different spatial positions and postures according to the personalized pelvic physiological structure and pelvic fracture type of the patients, thus achieving the stable clamping of the pelvis. By adopting the liquid expansion principle, the inner wall of the pin rod clamping part uniformly expands in an axial direction to guarantee the firm clamping of the pin and the uniform force on the pin and the cross rod. The butt joint module facilitates the rapid connection of the clamping device and the reduction robot. The clamping device has the obvious advantages of compact structure, flexible adjustment, large rigidity, firm fixation and easy assembly and disassembly and the like.

In conclusion, a universal and liquid expansion type clamping device for a pelvic fracture reduction robot includes an affected side spherical hinge support module, pin rod clamping parts, rod-rod clamping parts, a butt joint module, and a healthy side fixing module. The affected side spherical hinge support module is a main module for the pelvic clamping device, and by adopting a spherical hinge structure, the affected side spherical hinge support module is connected to the pin rod clamping part and the butt joint module, respectively. The pin rod clamping part, after firmly clamping pins at the anterior superior iliac spine and anterior inferior iliac spine on the affected side of the pelvis, is fixedly connected to cross rods of the affected side support; and the pin rod clamping parts, after firmly clamping the anterior superior iliac spine and anterior inferior iliac spine on the healthy side of the pelvis, are also fixedly connected to the healthy side cross rods. The rod-rod clamping parts are used to connect the healthy side cross rod and the healthy side adjustable support. The butt joint module is used to rapidly connect the affected side spherical hinge support module to a surgical robot. The pelvis on the healthy side is fixed by the fixing module, and after the pins placed into the pelvis on the healthy side and the cross rods are firmly clamped by the pin rod clamping parts, the firm fixation of the cross rods and the healthy side adjustable support is achieved using the rod-rod clamping parts. The adjustment flexibility of the clamping device is improved by adopting the spherical hinge structure in the affected side spherical hinge support module, a liquid expansion structure in the pin rod clamping part and multi-directional adjustment structures of the pin rod clamping part and rod-rod clamping part. The clamping device is able to adapt to pins at different spatial positions and postures according to the personalized pelvis and pelvic injury types of different patients, the adjustment is convenient, and stable clamping is achieved. The clamping device has obvious advantages of compact structure, flexible adjustment, high rigidity, firm fixation, easy assembly and disassembly and the like.

The embodiments of the present disclosure have been described with reference to the accompanying drawings, but the present disclosure is not limited to the above embodiments, and various changes may be made according to the inventive purpose of the present disclosure. All changes, modifications, substitutions, combinations or simplifications made according to the spiritual essence and principles of the technical solution of the present disclosure shall be equivalent substitutions, and shall fall within the scope of protection of the present disclosure as long as they conform to the inventive purpose of the present invention and do not depart from the technical principle and inventive idea of the present disclosure.

What is claimed is:

1. A universal and liquid expansion type clamping device for a pelvic fracture reduction robot, comprising:
an affected side spherical hinge support module;
a pin rod clamping part;
a rod-rod clamping part;
a butt joint module; and
a healthy side fixing module, wherein the affected side spherical hinge support module is a main module for the clamping device, and by adopting a spherical hinge structure, the affected side spherical hinge support module is connected to the pin rod clamping part and the butt joint module respectively, wherein the rod-rod clamping part is fixedly installed on one side of an operation table by the healthy side fixing module;
in which the adjustment flexibility of the clamping device is improved by adopting the spherical hinge structure in the affected side spherical hinge support module, a liquid expansion structure in the pin rod clamping part and multi-directional adjustment structures of the pin rod clamping part and rod-rod clamping part, the clamping device being able to adapt to pins at different spatial positions and postures according to personalized pelvis and pelvic injury types of different patients, wherein adjustment is convenient, and stable clamping is achieved.

2. The universal and liquid expansion type clamping device for the pelvic fracture reduction robot according to claim 1, wherein the affected side spherical hinge support module includes a spherical hinge base, a spherical hinge stop pin, a spherical hinge stopper, a spherical hinge cover plate and a spherical hinge cross rod, in which after the spherical hinge stop pin is placed in the spherical hinge base, the spherical hinge stopper can be adjusted by rotating the spherical hinge stop pin, wherein the spherical hinge stopper, after being adjusted by the spherical hinge stop pin, is able to limit the spherical hinge cross rod with the spherical hinge cover plate, wherein the spherical hinge cover plate is fixedly connected to the spherical hinge base, and in which adjustment flexibility of the clamping device is improved by adjusting an angle between the spherical hinge cross rod and the spherical hinge base by the affected side spherical hinge support module.

3. The universal and liquid expansion type clamping device for the pelvic fracture reduction robot according to claim 1, wherein the pin rod clamping part includes a pin, a bolt, a pin clamping assembly, a spring, a first cross rod clamping assembly, two compression bolts, and a nut, in which the pin clamping assembly is coaxially connected to the first cross rod clamping assembly by the bolt, wherein hydraulic oil is packaged in the pin clamping assembly, and the inner wall of the clamping part is expanded by screwing the compression bolts to achieve the effect of clamping the pin, wherein the first cross rod clamping assembly has basically the same structure as the bone needle clamping assembly and is used for clamping the cross bar, and the pin and the cross rod are fixed by screwing the bolt and the nut, in which a spring is arranged in a counterbore of a contact surface of the first cross bar clamping assembly and the pin clamping assembly such that during clamping, the clamping stability can be guaranteed under the combined action of a meshing contact of tooth grooves of the contact surface and the elastic potential energy of the spring.

4. The universal and liquid expansion type clamping device for the pelvic fracture reduction robot according to claim 1, wherein the rod-rod clamping part includes a bolt, a second cross rod clamping assembly, a spring, and a third cross rod clamping assembly, in which the second cross rod clamping assembly is coaxially connected to the third cross rod clamping assembly by a bolt, wherein the second cross rod clamping assembly is provided with textures at the joint with the cross rod to increase friction during clamping, wherein the third cross rod clamping assembly has basically the same structure as the second cross rod clamping assembly wherein during an operation, the medical staff is able to fix the cross rod by screwing the third cross rod clamping assembly and the bolt wherein a spring is arranged in a counterbore of the contact surface of the second cross rod clamping assembly and the third cross rod clamping assembly such that during clamping, clamping stability is guaranteed under the combined action of a meshing contact of tooth grooves of the contact surface and the elastic potential energy of the spring.

5. The universal and liquid expansion type clamping device for the pelvic fracture reduction robot according to claim 1, wherein the butt joint module includes a male connector, a female connector and two angular aluminum parts in which the male connector is fixed to the tail end of the fracture reduction robot by means of a threaded connection, in which the female connector is connected to the male connector in order to apply hydraulic pressure to the male connector for fixation, wherein the angular aluminum parts are fixed to the female connector and the affected side spherical hinge support module by means of a threaded connection; and in which the butt joint module is used for rapidly connecting the tail end of the fracture reduction robot to the affected side support to form a pelvic fracture reduction system.

6. The universal and liquid expansion type clamping device for the pelvic fracture reduction robot according to claim 1, wherein the healthy side fixing module includes two healthy side cross rods, a healthy side adjustable support, a healthy side fixing frame base, two healthy side fixing frame gaskets, two healthy side fixed frame rotary knobs, pin rod clamping parts and rod-rod clamping parts in which the healthy side cross rods are fixed to the healthy side adjustable support by the rod-rod clamping parts, in which the pins placed in the anterior superior iliac spine and anterior inferior iliac spine on the healthy side of the pelvis are fixed by the pin rod clamping parts, wherein the healthy side adjustable support is provided with equally spaced through holes and is able to be lifted and adjusted with the healthy side fixing frame base, and wherein the two healthy side fixing frame gaskets are pressed by the healthy side fixing frame base by rotating the healthy side fixing frame rotary knobs, such that the healthy side fixing frame base is fixed to the side of the operation table; and by adjusting the height of the healthy side adjustable support and an angle of the healthy side cross rod, the healthy side fixing module is able to adapt to pins at different spatial positions and postures on the healthy side of the pelvis.
